SEQUENCE LISTING (2 sequences)

SEQ ID NO: 1 - DNA, 20 bp, synthetic construct
  source 1..20: mol_type = other DNA
agagtttgatcmtggctcag

SEQ ID NO: 2 - DNA, 22 bp, synthetic construct
  source 1..22: mol_type = other DNA
tacggytaccttgttacgactt

(19) United States
(12) Patent Application Publication
(10) Pub. No.: US 2026/0308511 A1
(43) Pub. Date: Oct. 8, 2026

(54) STRAIN OF PAENIBACILLUS GLYCANILYTICUS AND APPLICATION THEREOF IN NITROGEN REMOVAL TREATMENT OF NITROGEN-CONTAINING AQUACULTURE WASTEWATER

(71) Applicants: Zhejiang Marine Fisheries Research Institute, Zhoushan (CN); Zhejiang University of Technology, Hangzhou (CN)

(72) Inventors: Lei Jin, Zhoushan (CN); Tiejun Li, Zhoushan (CN); Yin Zhu, Zhoushan (CN); Lei Qin, Hangzhou (CN); Yuanming Guo, Zhoushan (CN); Hui Shi, Zhoushan (CN)

(21) Appl. No.: 19/671,256

(22) Filed: May 8, 2026

Related U.S. Application Data
(63) Continuation of application No. PCT/CN2026/087902, filed on Apr. 1, 2026.

(30) Foreign Application Priority Data
Apr. 2, 2025 (CN) 202510404955.1

(51) Int. Cl. C12N 1/205; C02F 3/30; C02F 3/34; C02F 101/16; C12R 1/01

(52) U.S. Cl. CPC C12N 1/205; C02F 3/302; C02F 3/34; C02F 2101/16; C02F 2101/163; C02F 2305/06; C12R 2001/01

(57) ABSTRACT

A strain of Paenibacillus glycanilyticus and an application thereof in nitrogen removal treatment of nitrogen-containing aquaculture wastewater are provided, which belong to the technical field of nitrogen-containing aquaculture wastewater treatment. The strain of Paenibacillus glycanilyticus is Paenibacillus glycanilyticus DQ-1 and deposited at China Center for Type Culture Collection (CCTCC) with a deposit date of Jun. 13, 2024 and a deposit number of CCTCC NO: M 20241211. The strain of Paenibacillus glycanilyticus possesses heterotrophic nitrification-aerobic denitrification capability, and compared to other heterotrophic nitrifying-aerobic denitrifying bacteria, this strain exhibits a stronger nitrogen removal effect in a shorter period of time.

### TECHNICAL FIELD

The disclosure relates to the technical field of nitrogen-containing aquaculture wastewater treatment, and more particularly to a strain of Paenibacillus glycanilyticus and an application thereof in nitrogen removal treatment of nitrogen-containing aquaculture wastewater.

### STATEMENT REGARDING SEQUENCE LISTING

The sequence listing associated with this application is provided in text format in lieu of a paper copy and is hereby incorporated by reference into the specification. The name of the XML file containing the sequence listing is 26012THXT-USP1-US-2026-0010-SL.xml. The XML file is 2,926 bytes; is created on Apr. 9, 2026; and is being submitted electronically via patent center.

### BACKGROUND

In aquaculture, aquaculture ponds contain large amounts of fish and shrimp feces, pathological secretions, feed decomposition products, and dead organic matter. During the process of sedimentation and decomposition, these organic matters produce large quantities of inorganic nitrogen products, such as ammonia nitrogen, nitrate nitrogen, and nitrite nitrogen. These inorganic nitrogen compounds not only affect the health of aquatic animals but also, if discharged directly into the natural environment without treatment, can cause eutrophication of water bodies and damage the ecological environment. Therefore, nitrogen-rich aquaculture wastewater must undergo nitrogen removal treatment to meet discharge standards before being discharged into the natural environment.

Biological nitrogen removal utilizes the metabolic activities of microorganisms to reduce the concentration of nitrogen-containing pollutants in water. As a widely applied method for treating nitrogen-containing wastewater, it offers advantages such as good treatment efficiency, stable and reliable process performance, and convenient operation and management. Biological nitrogen removal wastewater treatment technology mainly reduces ammonia nitrogen in wastewater to nitrogen gas through the action of functional microbial groups such as ammonia-oxidizing bacteria, nitrifying bacteria, and denitrifying bacteria, thereby achieving the purpose of nitrogen removal. During the treatment process, nitrifying bacteria and nitrosifying bacteria convert ammonia nitrogen into nitrate nitrogen. Nitrate nitrogen has low chemical potential and high stability, which easily leads to nitrate accumulation. Denitrifying bacteria act on nitrate nitrogen in water, gradually reducing it to nitrite nitrogen, nitric oxide, and nitrous oxide, and finally to nitrogen gas released into the atmosphere, completing the nitrogen cycle. This is a key step in wastewater nitrogen removal. Traditional biological nitrogen removal is accomplished by the combined action of aerobic autotrophic nitrification by nitrifying bacteria and anaerobic heterotrophic denitrification by denitrifying bacteria. This process requires two separate reaction spaces, one aerobic and one anaerobic, for nitrification and denitrification respectively, thereby achieving nitrogen removal. Consequently, this treatment method occupies a large space, has high operating costs, and involves a relatively complicated process.

Until the 1980s, when a strain of aerobic denitrifying bacteria was first reported, aerobic denitrification technology was recognized and further promoted. Aerobic denitrifying bacteria are capable of using both oxygen and nitrate as electron acceptors in the presence of oxygen, simultaneously carrying out nitrification and denitrification reactions, thereby rapidly removing organic nitrogen. This technology has important application value in the field of biological nitrogen removal. After more than thirty years of research, a variety of aerobic nitrogen-removing microbial strains have been isolated and identified, exhibiting significant differences in growth characteristics and treatment capacity during heterotrophic nitrification-aerobic denitrification processes. Therefore, screening for efficient strains capable of simultaneous nitrification and denitrification has of great practical significance for nitrogen removal from polluted water bodies.

### SUMMARY

An objective of the disclosure is to provide a strain of Paenibacillus glycanilyticus. As a heterotrophic nitrifying-aerobic denitrifying bacterium, that is capable of performing nitrogen removal treatment on nitrogen-containing aquaculture wastewater and exhibits a superior ammonia nitrogen removal effect.

Technical solutions adopted by the disclosure to solve its technical problems are as follows.

In a first aspect, the disclosure provides a strain of Paenibacillus glycanilyticus, which is Paenibacillus glycanilyticus DQ-1. The strain of Paenibacillus glycanilyticus is deposited at China Center for Type Culture Collection (CCTCC) with a deposit date of Jun. 13, 2024, a deposit number of CCTCC NO: M 20241211, and the deposit address of Wuhan University, Wuhan, China.

The Paenibacillus glycanilyticus DQ-1 of the disclosure, a heterotrophic nitrifying-aerobic denitrifying bacterium, has the following biological characteristics. It is identified as a Gram-positive bacterium. Its colonies are white, opaque, circular, with a smooth and mucoid surface and neat, regular edges. Observation under a scanning electron microscope (SEM) shows that the cells are short rods, approximately (1.16-1.67) micrometers (μm)×(0.76-0.97) μm in size, and possess flagella and endospores. Optimal growth conditions for this strain are as follows: glucose as a carbon source, a carbon-to-nitrogen (C/N) ratio of 25, a pH of 7.0, and a temperature of 30° C.

In a second aspect, the disclosure provides a microbial agent including the strain of Paenibacillus glycanilyticus of the disclosure.

In an embodiment, the microbial agent further includes a pharmaceutically acceptable carrier, and the dosage form of the microbial agent is a solution, an emulsion, a suspension, a powder, a gel, a granule, or a lyophilized formulation.

In a third aspect, the disclosure provides an application of the strain of Paenibacillus glycanilyticus or the microbial agent of the disclosure in nitrogen removal treatment of nitrogen-containing aquaculture wastewater.

In an embodiment, the strain of Paenibacillus glycanilyticus acts as a heterotrophic nitrifying-aerobic denitrifying bacterium to perform nitrogen removal treatment on the nitrogen-containing aquaculture wastewater.

In an embodiment, the application specifically includes: inoculating the strain into the nitrogen-containing aquaculture wastewater, adding a carbon source (glucose or sucrose) to the nitrogen-containing aquaculture wastewater to adjust a C/N ratio of the nitrogen-containing aquaculture wastewater to 25, and culturing the strain in the nitrogen-containing aquaculture wastewater at pH 7.0 and 30° C. with shaking at a rotational speed of 121 revolutions per minute (rpm) to remove ammonia nitrogen, nitrate nitrogen, and total nitrogen from the wastewater.

In an embodiment, the strain is cultured under conditions of a C/N ratio of 25, a pH of 7, a temperature of 30° C., and a rotational speed of 121 rpm. The disclosure may achieve the following beneficial effects.

The Paenibacillus glycanilyticus DQ-1 of the disclosure, a heterotrophic nitrifying-aerobic denitrifying bacterium, is capable of utilizing glucose and sucrose as carbon sources. Under the conditions of glucose as the carbon source, a C/N mass ratio of 25, pH 7.0, a temperature of 30° C., and the rotational speed of 121 rpm, Paenibacillus glycanilyticus DQ-1 achieves an optimal nitrate nitrogen removal efficiency of 92.5% after 24 hours of aerobic cultivation, an optimal ammonia nitrogen removal efficiency of 93.3% after 48 hours of aerobic cultivation, and an optimal total nitrogen removal efficiency of 95.8% after 30 hours of aerobic cultivation. This has significant practical importance for the treatment of nitrogen-containing aquaculture wastewater. Regarding Paenibacillus glycanilyticus, it is the first discovery that a strain of this species possesses heterotrophic nitrifying-aerobic denitrifying capability. Compared to other heterotrophic nitrifying-aerobic denitrifying bacteria, this strain exhibits stronger nitrogen removal performance in a shorter period of time.

### BRIEF DESCRIPTION OF DRAWINGS

FIG. 1 illustrates a schematic diagram of morphological identification of heterotrophic nitrifying-aerobic denitrifying bacterium DQ-1 (i.e., Paenibacillus glycanilyticus DQ-1) according to the disclosure.

FIG. 2 illustrates a schematic diagram of a phylogenetic tree of the heterotrophic nitrifying-aerobic denitrifying bacterium DQ-1 based on 16S ribosomal deoxyribonucleic acid (rDNA) according to the disclosure.

FIG. 3 illustrates a schematic diagram of ammonia nitrogen degradation capability of the heterotrophic nitrifying-aerobic denitrifying bacterium DQ-1 according to the disclosure.

FIG. 4 illustrates a schematic diagram of nitrate nitrogen degradation capability of the heterotrophic nitrifying-aerobic denitrifying bacterium DQ-1 according to the disclosure.

FIG. 5 illustrates a schematic diagram of total nitrogen degradation capability of the heterotrophic nitrifying-aerobic denitrifying bacterium DQ-1 according to the disclosure.

### DETAILED DESCRIPTION OF EMBODIMENTS

Technical solutions of the disclosure are further described below through specific embodiments. It should be understood that the implementation of the disclosure is not limited to the following embodiments, and any modifications and/or changes in form made to the disclosure shall fall within the scope of protection of the disclosure.

In the disclosure, unless otherwise specified, all parts and percentages are by weight, and equipment and raw materials used are commercially available or commonly used in the art. Methods in the following embodiments are conventional methods in the art unless otherwise specified.

Unless otherwise specified, reagents used in the following embodiments can be purchased from conventional biochemical reagent suppliers.

Reagent MnSO₄·4H₂O in the following media is purchased from Shanghai Klin Biochemical Technology Co., Ltd., Luria-Bertani (LB) medium is purchased from Qingdao Hope Bio-Technology Co., Ltd., and all other chemical reagents are purchased from Sinopharm Chemical Reagent Co., Ltd.

### Embodiment 1: Screening and Identification of a Heterotrophic Nitrifying-Aerobic Denitrifying Bacterium

Ten grams (g) of sludge collected from a wastewater treatment pond at an aquaculture farm in Huzhou is added to an Erlenmeyer flask containing 100 milliliters (mL) of enrichment medium and incubated in a shaker at 28° C. and 120 rpm for 7-15 days. Then, the Erlenmeyer flask is transferred to a constant-temperature incubator at 28° C. and incubated for another 7-15 days. Subsequently, a small aliquot of the enriched culture is taken into a colorimetric tube, and Griess reagent is added to detect nitrite generation. To samples that turned red, 1 mL of 5 grams per liter (g/L) (NH₄)₂SO₄ solution is added for continued enrichment. Upon re-testing, when a result presented a deep red color, 1 mL of enrichment broth is transferred into 100 mL of isolation medium (i.e., liquid isolation medium) for continued incubation at a constant temperature. When the Griess reagent result indicates a deep red color, bacterial suspension is serially diluted and spread onto solid isolation medium plates by using the spread plate method. The plates are incubated in the constant-temperature incubator at 28° C. for 7 days. Pinpoint-sized single colonies are picked and streaked onto the solid isolation medium for isolation, then incubated at 28° C. This streaking process is repeated to obtain a pure bacterial strain, i.e., an isolated strain. To verify the nitrification ability of the isolated strain (also referred to as strain DQ-1), the isolated strain is inoculated into isolation medium (i.e., liquid isolation medium) containing 4% glucose and cultured at 28° C. and 120 rpm for 7 days, and the ammonia nitrogen content in culture broth (i.e., the isolation medium containing 4% glucose after cultivation) is determined by using a continuous flow injection method. To verify the denitrification capacity of the strain, the isolated strain is inoculated into 100 mL of denitrification medium supplemented with 5 g/L glucose and cultured on a shaker at 28° C. and 120 rpm for 24 hours. The nitrate content in culture broth (i.e., the denitrification medium supplemented with 5 g/L glucose after cultivation) is determined by the continuous flow injection method.

The enrichment medium (g/L) is formulated as follows: (NH₄)₂SO₄ 5 g, KH₂PO₄ 0.7 g, MgSO₄·7H₂O 0.5 g, CaCl₂) 0.36 g, distilled water 1 liter (L), pH 7.5. The liquid isolation medium (g/L) is formulated as follows: (NH₄)₂SO₄ 2 g, K₂HPO₄ 0.75 g, NaH₂PO₄ 0.25 g, MgSO₄·7H₂O 0.03 g, MnSO₄·4H₂O 0.01 g, CaCO₃ 5 g, distilled water 1 L, pH 7.0. The solid isolation medium is prepared by adding 1.8 g of agar to every 100 mL of liquid medium. The denitrification medium (g/L) is formulated as follows: NaNO₃ 0.1 g, KH₂PO₄ 0.7 g, MgSO₄·7H₂O 0.5 g, CaCl₂) 0.36 g, distilled water 1 L, pH 7.0. The LB medium (g/L) is formulated as follows: tryptone 10 g, yeast extract 5 g, NaCl 10 g. The solid medium is prepared by adding 1.8% agar powder to the liquid medium.

The isolated strain is streaked onto LB agar plates and then incubated in the constant-temperature incubator at a temperature of 30° C. for 48 hours to observe its colony characteristics. Bacterial cells of the isolated strain in the logarithmic growth phase are selected for Gram staining to determine the cell wall type. Subsequently, SEM is used to obtain high-resolution images of the bacterial cells to observe their detailed individual morphology. According to the guidelines of the Manual of Common Bacteriology and Bergey's Manual of Determinative Bacteriology, comprehensive physiological and biochemical identification of strain DQ-1 is performed.

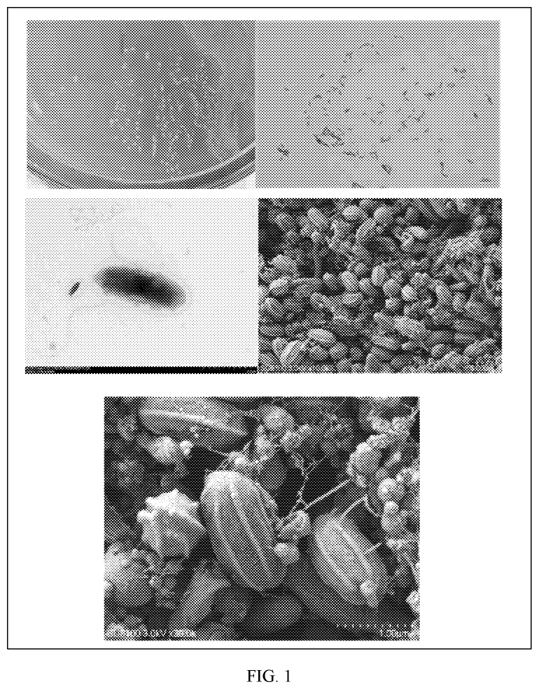

As shown in FIG. 1, colonies of the strain DQ-1 formed on LB solid medium are circular, with a smooth and mucoid surface, appearing white and opaque, and having neat, regular edges. Strain DQ-1 is Gram-positive. Observation under the SEM shows that cells of the strain DQ-1 are short rods, approximately (1.16-1.67) μm×(0.76-0.97) μm in size, and possess flagella and endospores.

The genomic DNA of strain DQ-1 is extracted using a genomic DNA extraction kit. Polymerase chain reaction (PCR) amplification is performed by using universal primers 27F: 5′-AGAGTTTGATCMTGGCTCAG-3′ (SEQ ID NO: 1) and 1492R: 5′-TACGGYTACCTTGTTACGACTT-3′ (SEQ ID NO: 2). The PCR product is verified by agarose gel electrophoresis (1.5%). After electrophoresis, the target bands are excised, purified, and sequenced. Sequencing is carried out by Sangon Biotech Co., Ltd. Reaction conditions of the PCR amplification are as follows: pre-denaturation at 94° C. for 5 minutes (min); denaturation at 94° C. for 1 min; annealing at 55° C. for 1 min; extension at 72° C. for 10 min; and final cooling at 4° C. for 10 min.

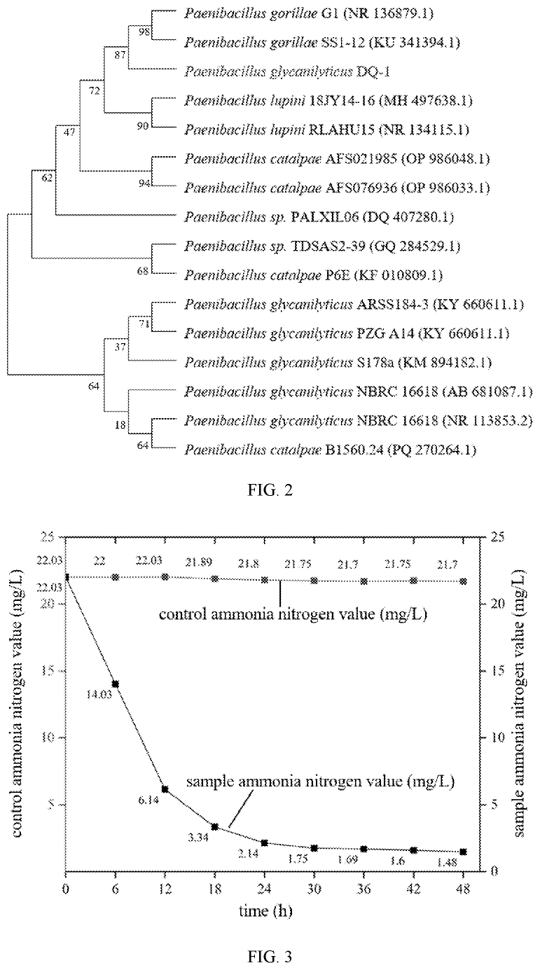

The 16S rDNA sequence of the strain DQ-1 is 1,449 base pairs (bp) in length. The sequence is uploaded to the National Center for Biotechnology Information (NCBI) database and aligned with existing bacterial 16S rDNA gene sequences in the NCBI database. The obtained alignment results show that the strain DQ-1 is in the same clade as Paenibacillus glycanilyticus. Based on colony morphological characteristics and physiological-biochemical identification results, strain DQ-1 is finally identified as Paenibacillus glycanilyticus. FIG. 2 shows a phylogenetic tree of the strain DQ-1.

The strain DQ-1, designated as Paenibacillus glycanilyticus DQ-1, is deposited at CCTCC, with a deposit number of CCTCC NO: M20241211 and a deposit address of Wuhan University, Wuhan, China.

Embodiment 2: identification of nitrification/denitrification capability of the strain DQ-1 in media with different nitrogen sources

A nitrification medium is formulated as follows: (NH₄)₂SO₄ 0.1 g, K₂HPO₄·3H₂O 0.75 g, NaH₂PO₄·2H₂O 0.25 g, NaCl 0.12 g, MnSO₄·H₂O 0.01 g, MgSO₄·7H₂O 0.01 g, FeSO₄·7H₂O 0.01 g, and distilled water to make up to 1000 mL, with a pH of 7.0-7.5.

A denitrification medium is formulated as follows: KNO₃ 0.1 g, K₂HPO₄·3H₂O 0.75 g, NaH₂PO₄·2H₂O 0.25 g, NaCl 0.12 g, MnSO₄·H₂O 0.01 g, MgSO₄·7H₂O 0.01 g, FeSO₄·7H₂O 0.01 g, and distilled water to make up to 1000 mL, with a pH of 7.0-7.5.

A simultaneous nitrification and denitrification medium is formulated as follows: (NH₄)₂SO₄ 0.1 g, KNO₃ 0.1 g, K₂HPO₄·3H₂O 0.75 g, NaH₂PO₄·2H₂O 0.25 g, NaCl 0.12 g, MnSO₄·H₂O 0.01 g, MgSO₄·7H₂O 0.01 g, FeSO₄·7H₂O 0.01 g, and distilled water to make up to 1000 mL, with a pH of 7.0-7.5.

Specifically, treatment methods for the above-mentioned media are as follows.

For the sample group, the seed solution of strain DQ-1 (the seed solution is a bacterial suspension cultured in LB liquid medium at 30° C. and 121 rpm for 24 hours) is inoculated at an inoculum size of 1% into nitrification medium, denitrification medium, and simultaneous nitrification and denitrification medium, respectively, and incubated in a shaker at 30° C. and 121 rpm.

For the control group, under the same conditions as the sample group, sterile water is inoculated at an inoculum size of 1% into nitrification medium, denitrification medium, and simultaneous nitrification and denitrification medium, respectively, and incubated in the shaker at 30° C. and 121 rpm.

In the nitrification medium group, samples are taken every 6 h to measure the ammonia nitrogen content. In the denitrification medium group, samples are taken every 3 h to measure the nitrate nitrogen content. In the simultaneous nitrification and denitrification medium group, samples are taken every 6 h to measure the total nitrogen content. Results are shown in FIGS. 3-5.

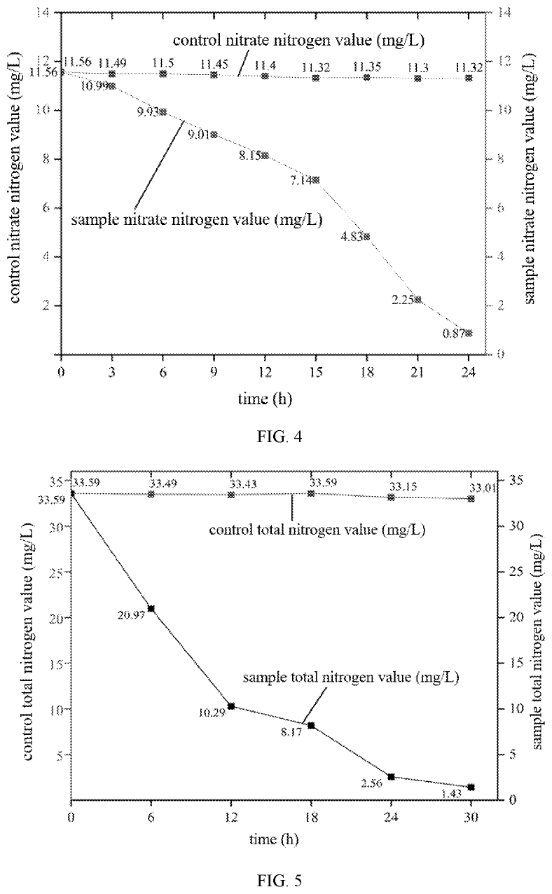

As shown in FIG. 3, under the culture conditions of 30° C. and 121 rpm, the strain DQ-1 reduces an ammonia nitrogen concentration from 22.03 milligrams per liter (mg/L) to 1.48 mg/L within 48 hours, with a degradation rate of 93.3%, indicating that the strain DQ-1 has good ammonia nitrogen degradation capability. As shown in FIG. 4, under the culture conditions of 30° C. and 121 rpm, the strain DQ-1 reduces a nitrate nitrogen concentration from 11.56 mg/L to 0.87 mg/L within 24 hours, with a degradation rate of 92.5%, indicating that the strain DQ-1 has good nitrate nitrogen degradation capability. As shown in FIG. 5, under the culture conditions of 30° C. and 121 rpm, the strain DQ-1 reduces a total nitrogen concentration from 33.59 mg/L to 1.43 mg/L within 30 hours, with a degradation rate of 95.8%, indicating that the strain DQ-1 has good total nitrogen degradation capability.

The above provides a detailed introduction to the strain of Paenibacillus glycanilyticus of the disclosure and its application in nitrogen removal treatment of nitrogen-containing aquaculture wastewater. Specific embodiments are used herein to illustrate the principles and implementation methods of the disclosure. The descriptions of the above embodiments are merely intended to facilitate understanding of the method and the core ideas of the disclosure. It should be noted that for those skilled in the technical field, without departing from the principles of the disclosure, several improvements and modifications may be made to the disclosure, and these improvements and modifications shall also fall within the protection scope of the appended claims of the disclosure.

1. A strain of Paenibacillus glycanilyticus, wherein the strain of Paenibacillus glycanilyticus is Paenibacillus glycanilyticus DQ-1 and deposited at China Center for Type Culture Collection (CCTCC) with a deposit date of Jun. 13, 2024 and a deposit number of CCTCC NO: M 20241211.

2. A microbial agent comprising the strain of Paenibacillus glycanilyticus as claimed in claim 1.

3. The microbial agent as claimed in claim 2, wherein the microbial agent further comprises a pharmaceutically acceptable carrier, and a dosage form of the microbial agent is a solution, an emulsion, a suspension, a powder, a gel, a granule, or a lyophilized formulation.

4. An application of the strain of Paenibacillus glycanilyticus as claimed in claim 1, comprising:
applying the strain of Paenibacillus glycanilyticus to nitrogen-containing aquaculture wastewater for nitrogen removal treatment.

5. An application of the microbial agent as claimed in claim 2, comprising:
applying the microbial agent to nitrogen-containing aquaculture wastewater for nitrogen removal treatment.

6. The application as claimed in claim 4, wherein the strain of Paenibacillus glycanilyticus acts as a heterotrophic nitrifying-aerobic denitrifying bacterium to perform nitrogen removal treatment on the nitrogen-containing aquaculture wastewater.

7. The application as claimed in claim 4, specifically comprising:
inoculating the strain of Paenibacillus glycanilyticus into the nitrogen-containing aquaculture wastewater, adding a carbon source to the nitrogen-containing aquaculture wastewater to adjust a carbon-to-nitrogen (C/N) ratio of the nitrogen-containing aquaculture wastewater to 25, and culturing the strain of Paenibacillus glycanilyticus in the nitrogen-containing aquaculture wastewater at a pH of 7.0 and a temperature of 30° C. with shaking at a rotational speed of 121 revolutions per minute (rpm) to remove ammonia nitrogen, nitrate nitrogen, and total nitrogen from the nitrogen-containing aquaculture wastewater, wherein the carbon source is glucose or sucrose.

8. The application as claimed in claim 4, wherein the strain of Paenibacillus glycanilyticus is cultured under conditions of a C/N ratio of 25, a pH of 7, a temperature of 30° C., and a rotational speed of 121 rpm.
